(19) United States
(12) Patent Application Publication
(10) Pub. No.: US 2024/0033194 A1
(43) Pub. Date: Feb. 1, 2024

(54) ANHYDROUS SOLID COMPOSITION COMPRISING A MAGNESIUM SALT AND STARCH

(71) Applicant: L’OREAL, Paris (FR)

(72) Inventors: Laure DAUBERSIES, Chevilly Larue (FR); Vincent THIRIOUX, Chevilly Larue (FR); Claudia BARBA, Chevilly Larue (FR); Fatma MEDARD, Saint Ouen (FR)

(21) Appl. No.: 18/258,066

(22) PCT Filed: Dec. 21, 2021
(86) PCT No.: PCT/EP2021/087090

§ 371 Date: Jun. 16, 2023

(30) Foreign Application Priority Data
Dec. 22, 2020 (FR) 2013892

(51) Int. Cl. A61K 8/22; A61K 8/02; A61Q 15/00

(52) U.S. Cl. CPC A61K 8/22; A61K 8/0216; A61Q 15/00; A61K 2800/31; A61K 2800/34; A61K 2800/87

(57) ABSTRACT

The present invention relates to an anhydrous solid cosmetic composition, particularly for treating body odors resulting from perspiration, comprising, in a cosmetically acceptable medium, from 5% to 25% by weight with respect to the total weight of the composition of at least one magnesium salt and from 15% to 40% by weight with respect to the total weight of the composition of starch.

Figure for the abstract: none

The present invention relates to an anhydrous solid cosmetic composition, particularly for treating body odors resulting from perspiration, which comprises at least one magnesium salt and starch.

In the field of cosmetics, the use in topical application of deodorant products containing deodorant type active substances for reducing or eliminating body odors, in particular underarm odors, which are generally unpleasant, is well known.

Eccrine or apocrine sweat generally has a low odor when secreted. It is the degradation thereof by bacteria via enzyme reactions that produces malodorous compounds. The function of deodorant agents is thus that of reducing or preventing the formation of unpleasant odors. This aim can be achieved particularly via a deodorant activity.

The different systems proposed to date can be grouped into major families. A first family relates to unpleasant odor absorbers. These absorbers “capture” or reduce the volatility of odorous compounds. Bactericidal substances are also known, preferably selective in respect of the strains responsible for odors, or limiting bacterial growth. Of the bactericidal substances destroying the resident bacterial flora, that most used is Triclosan (2,4,4′-trichloro-2′-hydroxydiphenylether). Of the substances reducing bacterial growth, mention may be made of transition metal chelating agents such as EDTA or DPTA. Substances inhibiting enzyme reactions responsible for the formation of odorous compounds are also known, particularly arylsulfatase, 5-lipoxygenase, aminocyclase, or p-glucoronidase inhibitors; A deodorant activity can also be obtained by neutralizing the volatile compounds responsible for the odor.

Finally, aluminum and/or zirconium salts are also used as antibacterials. These salts play a direct role in deodorant efficacy by reducing the number of bacteria responsible for the degradation of sweat. However, these different treatments applied on the skin of the armpits tend to cause skin alterations.

Deodorant products are in general available in the form of roll-on, sticks, aerosol or spray. The most effective dosage forms for combatting unpleasant odor are alcoholic dosage forms. However, they have the drawback of causing discomfort during application, in particular after shaving the armpit. Emulsions have the drawback of being wetting and having difficulty drying under the armpit. Anhydrous sticks and aerosols are oily dosage forms which leave an oily texture under the armpit, and some of which tend to transfer onto clothing, leaving visible and unsightly stains.

In order to remedy the problem of stains in particular, deodorant compositions containing oils having a refractive index similar to those of aluminum salts have been developed. The role of such oils is to reduce the whitish appearance of the compositions when they are applied on the skin, and, hence, render the stains on clothing less white.

However, such deodorant compositions have the drawback of giving the skin, particularly at the armpits, an unpleasant oily sensation for the user, and do not help limit the transfer of deodorant products from the skin onto clothing.

More natural deodorant compositions free from aluminum salts are already proposed, but it is found that the deodorant efficacy thereof does not generally exceed 4 hours following application.

Therefore, there is a constant need for a composition for treating body odors associated with human perspiration, particularly underarm odors, which does not have all of the drawbacks cited above. Compositions making it possible to do away with the use of aluminum salts and/or aluminum and zirconium complexes, while providing deodorant efficacy greater than 24 hours, or even greater than 48 hours, are now particularly sought.

The aim of the invention is particularly to respond to these expectations.

It is more specifically aimed at proposing solid cosmetic compositions, particularly for treating body odors resulting from perspiration, not requiring the presence of aluminum salts and which prove to be just as effective. Indeed, the applicant surprisingly discovered that all of the aims cited above can be achieved by the composition according to the present invention. Such compositions are furthermore stable, at least at ambient temperature and at 4° C. for at least 2 months.

According to a first aspect, the present invention thus relates to an anhydrous solid cosmetic composition, particularly for treating body odors resulting from perspiration, comprising, in a cosmetically acceptable medium, from 5% to 25% by weight with respect to the total weight of the composition of at least one magnesium salt and from 15% to 40% by weight with respect to the total weight of the composition of starch.

Associating magnesium salt(s) and a high starch content (i.e., from 15% to 40% by weight) in an anhydrous solid composition, for example an anhydrous stick, according to the invention makes it possible to limit the formation of malodorous underarm odors for a much longer period than the solid compositions currently available, particularly for at least 24 h, preferably at least 48 h.

The composition according to the invention thus has a performance at least equal to solid compositions comprising a large quantity (i.e., at least 12% by weight) of aluminum salts. However, the composition according to the invention does not require the presence of aluminum salts.

Furthermore, the solid composition according to the invention enables easy and sufficient deposition of composition in a single passage on the skin, with no tacky effect, and does not cause irritation.

Preferably, the anhydrous solid composition according to the invention comprises less than 5% by weight of aluminum salt(s), preferably less than 3% by weight, preferably less than 1% by weight.

Preferably, the solid composition according to the invention is totally free from aluminum salt(s).

The term “deodorant agent” or “deodorant active agent” denotes any substance or any composition capable of substantially reducing, or even eliminating, unpleasant odors, in particular body odor, in particular unpleasant odor resulting from the decomposition of human sweat by bacteria.

The term “cosmetic method for treating body odors resulting from perspiration” denotes a method which, used on human skin, substantially reduces, or even eliminates, unpleasant odors, in particular body odor, in particular unpleasant odor resulting from the decomposition of human sweat by bacteria.

The term “cosmetically acceptable medium” denotes a medium compatible with the skin and/or appendages or mucosa thereof, having a pleasant color, odor and texture and not giving rise to unacceptable discomfort (such as tightness), liable to dissuade the consumer from using the composition. Said cosmetically acceptable medium is also a medium that does not leave any traces during the application thereof, and that does not as such stain clothing. Said cosmetically acceptable medium is anhydrous.

The term “anhydrous” denotes a composition comprising less than 1% by weight of water with respect to the total weight of the composition, preferably less than 0.5% by weight, preferably less than 0.1% by weight. Preferably, an anhydrous composition is a totally water-free composition.

### Solid Composition

The term “solid composition” denotes that the measurement of the maximum force measured by means of texturometric analysis on inserting a probe in the sample of formula should be at least equal to 6 Newton, in particular at least equal to 7 Newton, particularly at least equal to 9 Newton, assessed under precise measurement conditions as follows.

The texturometric measurements are made on the formulas packaged in the plastic or cardboard packaging thereof (finished products). The hardness of the formulas is measured after leaving the finished products to stand for 2 hours at 25° C. and at 40% hygrometry. The equipment consists of a shearing device coupled with a force sensor, with relative movement at controlled speed; the texturometer is TA.XTplus Texture Analyzer sold by Stable Micro Systems, and the protocol is as follows:

The hardness corresponds to the force required, in Newton, for the punch to penetrate the sticks by 35 mm. The tests are repeated between 3 times per product.

A “cutting wire” type punch is brought into contact with the sample at a speed of 0.5 mm/s. The measurement system detects the interface with the sample with a detection threshold equal to 0.005 Newton. The probe is inserted 35 mm into the sample, at a rate of 0.5 mm/s. The measurement apparatus records the progression of the compression force measured over time, during the penetration phase. The hardness of the sample corresponds to the mean of the maximum values of the force detected during penetration, for 3 measurements.

Preferably, the solid composition according to the invention is presented in stick form.

### Magnesium Salt

The anhydrous solid cosmetic composition according to the invention comprises from 5% to 25% by weight with respect to the total weight of the composition of at least one magnesium salt.

When the magnesium salt content is greater than 25% by weight, the deposition is much less satisfactory and the solid composition becomes too hard and thus has a risk of breaking. Furthermore, it is also difficult to manufacture.

Preferably, the magnesium salt is chosen from magnesium oxide, magnesium carbonate, magnesium hydroxide, magnesium bicarbonate and mixtures thereof. More preferably, the magnesium salt is magnesium oxide. Magnesium oxide is the compound having the formula MgO.

Preferably, the magnesium salt is present in a content ranging from 7 to 20% by weight, preferably from 8 to 15%, preferably from 9 to 12% by weight, with respect to the total weight of the composition. Obviously, the content is expressed in active substance.

### Starch

The anhydrous solid cosmetic composition according to the invention comprises from 15% to 40% by weight with respect to the total weight of the composition of starch.

Preferably, the starch is chosen from corn, tapioca, rice or wheat starch. It can be modified or non-modified (also known as “native”). Preferably, the starch is native. Preferably, the starch is native corn starch.

Preferably, the starch is present in a content ranging from 18% to 30%, preferably from 20% to 28%, preferably from 21% to 25%, by weight with respect to the total weight of the composition. Obviously, the content is expressed in active substance.

30 Preferably, the weight ratio between the magnesium salt(s) and the starch is less than or equal to 1, preferably between 0.3 and 0.6, preferably between 0.3 and 0.5.

### Oily Phase

The anhydrous solid composition according to the invention also comprises a cosmetically acceptable medium, which is therefore anhydrous. This medium corresponds to an oily phase.

The term oily phase denotes an organic solid or liquid phase that is non-miscible in water. This generally includes one or more hydrophobic compounds rendering said phase non-miscible in water. Said phase can be solid or liquid (in the absence of a structuring agent) at ambient temperature (20-25° C.).

Preferably, the composition according to the invention comprises at least one solid fat chosen preferably from waxes, pasty fats, and mixtures thereof.

### Waxes

Preferably, the composition according to the invention comprises at least one wax.

Generally, the wax considered within the scope of the present invention is a lipophilic compound, which is solid at ambient temperature (25° C.), having a reversible solid/liquid change of state and a melting point greater than or equal to 30° C. of up to 200° C. and particularly up to 120° C. In particular, the waxes suitable for the invention may have a melting point greater than or equal to 45° C., and particularly greater than or equal to 55° C. The waxes suitable for use in the compositions according to the invention are chosen from animal, plant, mineral or synthetic waxes, which are solid at ambient temperature, and mixtures thereof.

Mention can be made for example the following hydrocarbon waxes comprising a fatty alkyl chain having in general from 10 to 60 carbon atoms, preferably from 20 to 40 carbon atoms, said chain able to be saturated or unsaturated, substituted or not, linear, branched or cyclic, preferably saturated and linear:
fatty alcohols, preferably C12-C22;
esters of fatty alcohols;
fatty acids;
fatty acid amides;
fatty acid esters including triglycerides;
fatty acid ethers;
ethoxylated fatty alcohols;
ethoxylated fatty alcohols, and the corresponding salts thereof.

Preferably, the composition according to the invention comprises at least one fatty alcohol, preferably C12-C22. Of the fatty alcohols, mention can be made of lauric, cetyl, stearyl, cetearyl, oleic, behenic, linoleic, undecylenic, palmitoleic, linolenic, arachidonic, erucic alcohols, and mixtures thereof. Preferably, the fatty alcohol is cetearyl alcohol (i.e., which is a mixture of fatty alcohols essentially composed of cetyl and stearyl alcohols). In the compositions according to the invention, the fatty alcohol content can vary from 10 to 30% by weight, and preferably from 10 to 25%, more preferably from 10 to 22% by weight with respect to the total weight of the composition. In particular, these fatty alcohols act as crystallizing structuring fatty acids. The maximum content of these fatty alcohols in the composition is advantageously 30%, as beyond this content, it becomes difficult to deposit the composition during the application of the composition on the skin. Preferably, a fatty alcohol content ranging from 10 to 22% by weight allows a better compromise both in terms of the structuring and stability properties of the composition, while enabling sufficient deposition of composition during application on the skin.

Among the esters of fatty alcohols, mention can be made of tri-isostearyl citrate, ethyleneglycol-di-12-hydroxystearate, tristearylcitrate, stearyl octanoate, stearyl heptanoate, trilauryl citrate and mixtures thereof.

Among the fatty acid esters, mention can be made of ester waxes, monoglycerides, diglycerides, or triglycerides.

As an ester wax, mention can be made of stearyl stearate, stearyl behenate, stearyl octyldodecanol, cetearyl behenate, behenyl behenate, ethyleneglycol, distearate, ethyleneglycol dimaplimitate. It is possible to use in particular a C20-C40 alkyl (hydroxystearyloxy)stearate (the alkyl group comprising 20 to 40 carbon atoms), alone or in a mixture, may be used as the wax.

Among the triglyceride waxes, mention can be made more particularly of tribehenin, C18-C36 triglyceride, and mixtures thereof.

By way of illustration of waxes suitable for the invention, particular mention may be made of hydrocarbon waxes such as beeswax, lanolin wax, and Chinese insect waxes, rice bran wax, carnauba wax, candelilla wax, ouricury wax, alfa wax, berry wax, shellac wax, Japan wax and sumac wax; montan wax, microcrystalline waxes, paraffins and ozokerite; polyethylene waxes, waxes obtained by means of Fisher-Tropsch synthesis and waxy copolymers and the esters thereof.

Mention may also be made of waxes obtained by means of the catalytic hydrogenation of animal or plant oils having C8-C32 linear or branched fat chains. Of these, particular mention may be made of hydrogenated sunflower oil, hydrogenated castor oil, hydrogenated coconut oil, hydrogenated lanolin oil, and di-(trimethylol-1,1,1 propane) tetrastearate sold under the trade name Hest 2T-4S® by Heterene.

Mention may also be made of silicone waxes (C30-45 Alkyl dimethicone) and fluorinated waxes. It is also possible to use waxes obtained by hydrogenating esterified castor oil with cetyl alcohol sold under the trade names Phytowax ricin 16L64® and 22L73® by Sophim. Such waxes are in particular described in the application FR 2 792 190.

The composition according to the invention preferably comprises at least one plant-based wax. Preferably, the composition according to the invention comprises at least one wax chosen from hydrocarbon waxes such as beeswax, rice bran wax, carnauba wax, candelilla wax, ouricury wax, alfa wax, berry wax, shellac wax, Japan wax and sumac wax, montan wax; hydrogenated sunflower oil, hydrogenated castor oil, hydrogenated coconut oil, hydrogenated lanolin oil, and di-(trimethylol-1,1,1 propane) tetrastearate. Preferably, the composition according to the invention comprises at least one such wax in a content ranging from 3% to 15% by weight, and in particular from 4% to 10% by weight with respect to the total weight of the composition.

Preferably, the composition according to the invention comprises at least one fatty alcohol, preferably C12-C22, and at least one plant-based wax.

### Pasty Fats

Preferably, the composition according to the invention comprises at least one pasty fat.

For the purposes of the invention, the term “pasty fat” denotes a lipophilic fatty compound having a reversible solid/liquid change of state, having an anisotropic crystalline organization in the solid state, and including a liquid fraction and a solid fraction at a temperature of 23° C.

In other words, the initial melting point of the pasty compound may be less than 23° C. The liquid fraction of the pasty compound measured at 23° C. may represent 9% to 97% by weight of the compound. This liquid fraction at 23° C. preferably represents between 15% and 85%, more preferably between 40% and 85% by weight. According to the invention, the melting point is equivalent to the temperature of the most endothermic peak observed in thermal analysis (DSC) as described in the standard ISO 11357-3: 1999. The melting point of a paste or a wax may be measured using a differential scanning calorimeter (DSC), for example the calorimeter sold under the name “MDSC 2920” by 45 TA Instruments.

The measurement protocol is as follows: a 5 mg sample of paste or wax (according to the case) placed in a crucible is subjected to a first temperature rise from −20° C. to 100° C., at a heating rate of 10° C./minute, and is then cooled from 100° C. to −20° C. at a cooling rate of 10° C./minute and finally subjected to a second temperature rise from −20° C. to 100° C. at a heating rate of 5° C./minute. During the second temperature rise, the variation in the difference in power absorbed by the empty crucible and by the crucible containing the paste or wax sample as a function of temperature is measured.

The melting point of the compound is the value of the temperature equivalent to the top point of the peak of the curve representing the variation in the difference in power absorbed as a function of temperature.

The liquid fraction by weight of the pasty compound at 23° C. is equal to the ratio of the enthalpy of fusion consumed at 23° C. to the enthalpy of fusion of the pasty compound. The enthalpy of fusion of the pasty compound is the enthalpy consumed by the compound to change from the solid state to the liquid state. The pasty compound is said to be in the solid state when the entire mass thereof is in solid crystalline form. The pasty compound is said to be in the liquid state when the entire mass thereof is in liquid form.

The enthalpy of fusion of the pasty compound is equal to the area under the curve of the thermogram obtained using differential scanning calorimeter (DSC), such as the calorimeter sold under the trade name MDSC 2920 by TA instrument, with a temperature rise of 5° C. or 10° C. per minute, according to the ISO 11357-3:1999 standard. The enthalpy of fusion of the pasty compound is the quantity of energy required to change the compound from the solid state to the liquid state. It is expressed in J/g. The enthalpy of fusion consumed at 23° C. is the quantity of energy required by the sample to change from the solid state to the state presented at 23° C. consisting of a liquid fraction and a solid fraction.

The liquid fraction of the pasty compound measured at 32° C. preferably represents 30% to 100% by weight of the compound, preferably 50% to 100%, more preferably 60% to 100% by weight of the compound. If the liquid fraction of the pasty compound measured at 32° C. is equal to 100%, the temperature of the end of the melting range of the pasty compound is less than or equal to 32° C.

The liquid fraction of the pasty compound at 32° C. is equal to the ratio of the enthalpy of fusion consumed at 32° C. to the enthalpy of fusion of the pasty compound. The enthalpy of fusion consumed at 32° C. is calculated as for the enthalpy of fusion consumed at 23° C.

The pasty compound preferably has a hardness at 20° C. ranging from 0.001 to 0.5 MPa, preferably from 0.002 to 0.4 MPa.

The hardness is measured according to a probe penetration method in a sample of compound and in particular using a texture analyzer (for example TA-XT2i from Rheo) equipped with a 2 mm diameter stainless steel cylinder. The hardness measurement is performed at 20° C. at the center of 5 samples. The cylinder is inserted into each sample, the penetration depth being 0.3 mm. The hardness value recorded is that of the maximum peak.

The pasty compound is preferably chosen from plant-based compounds.

The pasty compound can be chosen particularly from isomerized jojoba oil such as the trans isomerized partially hydrogenated jojoba oil manufactured or sold by Desert Whale under the trade name !so-Jojoba-500, orange wax such as, for example, that marketed under the trade name Orange Peel Wax by Koster Keunen, cupuacu butter (Rain forest RF3410 from Beraca Sabara), shorea butter, murumuru butter (RAIN FOREST RF3710 from Beraca Sabara), shea butter, partially hydrogenated olive oil such as, for example, the compound marketed under the trade name Beurrolive by Soliance, cocoa butter, mango oil such as, for example, Lipex 203 from Aarhuskarlshamn and mixtures thereof.

Preferably, he pasty compound is chosen from isomerized jojoba oil, orange wax, cupuacu butter, shea butter, partially hydrogenated olive oil, cocoa butter, mango butter, shorea butter, murumuru butter, and mixtures thereof.

Advantageously, the compound according to the invention comprises a pasty plant-based compound according to a content by weight within the range from 1 to 6%, preferably from 2 to 5%, preferably from 3 to 4% by weight, with respect to the total weight of the composition.

### Oil

The composition according to the invention preferably comprises at least one oil.

The term “oil” denotes a fat that is liquid at ambient temperature (25° C.) and atmospheric pressure (760 mm Hg namely 10⁵ Pa). The oil may be volatile or non-volatile.

The term “volatile oil” according to the invention denotes any oil capable of evaporating in contact with skin or keratin fiber in less than one hour, at ambient temperature and atmospheric pressure. The volatile oils according to the invention are volatile cosmetic oils that are liquid at ambient temperature, having a vapor pressure different to zero, at ambient temperature and atmospheric pressure, particularly ranging from 0.13 Pa to 40,000 Pa (10⁻³ to 300 mm Hg), particularly ranging from 1.3 Pa to 13,000 Pa (0.01 to 100 mm Hg), and more specifically ranging from 1.3 Pa to 1300 Pa (0.01 to 10 mm Hg).

The term “non-volatile oil” denotes an oil remaining on skin or keratin fiber at ambient temperature and atmospheric pressure for at least several hours and particularly having a vapor pressure less than 10⁻³ mm Hg (0.13 Pa).

The oil may be chosen from any physiologically acceptable and particularly cosmetically acceptable oils, in particular mineral, animal, plant, synthetic oils; in particular, volatile or non-volatile hydrocarbon and/or silicone and/or fluorinated oils and mixtures thereof.

More specifically, the term “hydrocarbon oil” denotes an oil essentially comprising carbon and hydrogen atoms and optionally one or more functions chosen from hydroxyl, ester, ether, carboxylic functions. Generally, the oil has a viscosity of 0.5 to 100,000 mPa·s, preferably from 50 to 50,000 mPa·s and more preferably from 100 to 300,000 mPa·s.

By way of examples of volatile oils suitable for use in the invention, mention may be made of:
volatile hydrocarbon oils chosen from hydrocarbon oils having 8 to 16 carbon atoms, and particularly petroleum-based C₈-C₁₆ isoalkanes (also referred to as isoparaffins) such as isododecane (also referred to as 2,2,4,4,6-pentamethylheptane), isodecane, isohexadecane, and for example the oils sold under the trade names Isopars or Permetyls, C₈-C₁₆ branched esters, iso-hexyl neopentanoate, and mixtures thereof. Further volatile hydrocarbon oils such as petroleum distillates, particularly those sold under the trade name Shell Solt by SHELL, may also be used; volatile linear alkanes such as those described in the Cognis patent application DE10 2008 012 457.
and mixtures thereof.

Preferably, the composition according to the invention comprises at least one non-volatile oil. By way of examples of non-volatile oils suitable for use in the invention, mention may be made of:
plant-based hydrocarbon oils such as liquid fatty acid triglycerides having 4 to 24 carbon atoms such as heptanoic or octanoic triglycerides or plant oils such as wheat germ, olive oils, sweet almond, palm, rapeseed, cotton, coconut, alfalfa, poppy seed, pumpkin, squash, blackcurrant seed, evening primrose, millet, barley, quinoa, rye, safflower, candlenut, passiflora, musk rose, sunflower, corn, soybean, squash, grape seed, sesame, hazelnut, apricot, macadamia, castor, avocado oils, fractionated coco-caprylic/capric acid oil, caprylic/capric acid triglycerides such as those sold by Stearineries Dubois or those sold under the trade names Miglyol 810, 812 and 818 by SASOL;
linear or branched hydrocarbons of mineral or synthetic origin, such as liquid paraffins and derivatives thereof, petroleum jelly, polydecenes, polybutenes, hydrogenated polyisobutene such as Parleam, squalane;
synthetic ethers having from 10 to 40 carbon atoms;
synthetic esters particular of fatty acids such as the oils having the formula R₁COOR₂ wherein R₁ represents the residue of a linear or branched higher fatty acid comprising 1 to carbon atoms and R₂ represents a hydrocarbon chain, particularly branched containing 1 to 40 carbon atoms with R₁+R₂ is greater than or equal to 10 such as for example Purcellin oil (cetostearyl octanoate)), isononyl isononanoate, isopropyl myristate, isopropyl palmitate, isoamyl laurate, C₁₂ to C₁₅ alcohol benzoate, hexyl laurate, diisopropyl adipate, isononyl isononanoate, 2-ethylhexyl palmitate, octyl-2-dodecyl stearate, octyl-2-dodecyl erucate, isostearyl isostearate, tridecyl trimellitate; octanoates, decanoates or ricinoleates of alcohols or polyalcohols such as propylene glycol dioctanoate; hydroxylated esters, such as isostearyl lactate, octyl hydroxy stearate, octyl dodecyl hydroxy stearate, diisostearyl-malate, triisocetyl citrate; heptanoates, octanoates, decanoates of fatty alcohols; polyol esters, such as propylene glycol dioctanoate, neopentyl glycol diheptanoate and diethylene glycol diisononanoate; and pentaerythritol esters such as pentaerythrityl tetraisostearate;
   fatty alcohols that are liquid at ambient temperature, with a branched and/or unsaturated carbon chain having 12 to 26 carbon atoms, such as octyldodecanol, isostearyl alcohol, 2-butyloctanol, 2-hexyl decanol, 2-undecylpentadecanol, oleic alcohol;
   higher fatty acids, such as oleic acid, linoleic acid, linolenic acid;
   fluorinated oils optionally partially hydrocarbon-based and/or silicone-based, such as fluorosilicone oils, fluorinated polyethers or fluorinated silicones, as described in document EP-A-847 752;

silicone oils, such as polydimethylsiloxanes (PDMS) which are non-volatile and linear or cyclic; polydimethylsiloxanes comprising alkyl, alkoxy or phenyl groups which are pendant or at the end of the silicone chain, said groups having from 2 to 24 carbon atoms; phenylated silicones, such as phenyl trimethicones, phenyl dimethicones, phenyltrimethylsiloxydiphenylsiloxanes, diphenyl dimethicones, diphenylmethyldiphenyl-trisiloxanes or (2-phenylethyl)trimethylsiloxysilicates; and
mixtures thereof.

Preferably, the composition according to the invention comprises at least one non-volatile oil chosen from plant oils and particularly wheat germ, olive oils, sweet almond, palm, rapeseed, cotton, coconut, alfalfa, poppy seed, pumpkin, squash, blackcurrant seed, evening primrose, millet, barley, quinoa, rye, safflower, candlenut, passiflora, musk rose, sunflower, corn, soybean, squash, grape seed, sesame, hazelnut, apricot, macadamia, castor, avocado oils, fractionated coco-caprylic/capric acid oil and caprylic/capric acid triglycerides; and ester oils of formula R₁COOR₂ wherein R₁ represents the residue of a linear or branched fatty acid including from 1 to 40 carbon atoms and R₂ represents a hydrocarbon chain, particularly branched, containing from 1 to 40 carbon atoms where R₁+R₂ is greater than or equal to 10 such as Purcellin oil, isononyl isononanoate, isopropyl myristate, isopropyl palmitate, isoamyl laurate, C₁₂ to C₁₅ alcohol benzoate, hexyl laurate, diisopropyl adipate, isononyl isononanoate, ethyl 2-hexyl palmitate, octyl 2-dodecyl stearate, octyl 2-dodecyl erucate, isostearyl isostearate or tridecyl trimellitate.

Advantageously, the composition according to the invention comprises at least one oil, preferably non-volatile, preferably chosen from plant oils and ester oils of formula R₁COOR₂ cited above, according to a content by weight between 20% and 80%, preferably from 25 to 60%, preferably from 30 to 50% by weight, with respect to the total weight of the composition.

Preferably, the composition is substantially free from silicone oil. The term “substantially free” denotes that the composition comprises less than 3% by weight, preferably less than 1% by weight, preferably less than 0.5% by weight, preferably less than 0.3% by weight, preferably less than 0.1% by weight of silicone oil with respect to the total weight of the composition. Preferably, the composition is totally free from silicone oil.

### Additives

The composition according to the invention can also comprise cosmetic additives chosen from among opacifiers, stabilizers, preservatives, perfume, solar filters, cosmetic active agents, suspension agents, sequestrants, dyes or any other ingredient routinely used in cosmetics for this type of application.

Obviously, a person skilled in the art will take care to choose this or these optional additional compound(s) in such a way that the advantageous properties intrinsically associated with the composition according to the invention are not altered, or are not substantially altered, by the envisaged additive(s).

The present invention also relates to a cosmetic method for treating body odors resulting from perspiration, comprising the application of the composition according to the invention on the skin.

Finally, the present invention also relates to a product comprising an anhydrous solid composition according to the invention, packaged in a container which can be made of plastic, cardboard or paper. The container, also referred to as “packaging”, contains the composition. It is preferably made of plastic or cardboard. The product is the finished product, with contains the stick in the container thereof.

The expressions “between . . . and . . . ” and “ranging from . . . to . . . ” are to be understood to be inclusive of the limits, unless specified otherwise.

In the description and the examples, unless mentioned otherwise, the percentages are weight percentages. The temperature is expressed in degrees Celsius unless mentioned otherwise, and the pressure is the atmospheric pressure, unless mentioned otherwise.

The invention is illustrated in more detail by the non-limiting examples presented hereinafter.

The examples that follow are used to illustrate this invention. The quantities are indicated as percentages by weight with respect to the total weight of the composition (% w/w).

### EXAMPLE 1

### Anhydrous Solid Composition According to the Invention and Deodorant Performance Evaluation

1/ Anhydrous solid composition a according to the invention was prepared according to the following protocol:

Place the oily phase (phase A) in a beaker and place it in a water-bath at a set-point temperature of 100° C. until the fats have completely melted;

Set the set-point temperature of the melter to 90° C. After introducing phase A into the melter, set the rotor-stator to 400-500 rpm and mix;

Gradually add magnesium oxide (phase B) under stirring at 600 rpm for about 5 min, and allow to homogenize for 10 min;

Gradually add corn starch (phase C) under stirring at 800 rpm for about 5 min, and allow to homogenize for 5 min;

Stop stirring with the rotor-stator and place the mixture under stirring with a bar magnet; pour the sticks at a set-point temperature of 68-70° C.;

Place the sticks at 4° C. for 45 min until fully solidified.

TABLE 1

| | A | |
|---|---|---|
| Ingredient | % (w/w) | Phase |
| CORN STARCH | 22 | C |
| MAGNESIUM OXIDE | 10 | B |
| CETYLSTEARYL ALCOHOL | 15 | A |
| Caprylic/capric triglycerides | Qs 100 | A |
| REFINED RAPESEED OIL | — | A |
| HYDROGENATED CASTOR OIL | 4 | A |
| SHEA BUTTER | 4 | A |

2/ Formula A is compared to comparative compositions C1 (containing aluminum salts) and C2 below.

TABLE 2

| | C1 | C2 |
|---|---|---|
| | (comparative) | (comparative) |
| Ingredient | % (w/w) | % (w/w) |
| MAGNESIUM OXIDE | — | 7 |
| CETYLSTEARYL ALCOHOL | 25 | 25 |
| C12-C15 alkyl benzoate | Qs 100 | Qs 100 |
| Isopropyl myristate | 26.7 | 26.7 |
| HYDROGENATED CASTOR OIL | 4 | 8 |
| Perlite | 0.2 | — |
| Aluminum salts | 15 | — |
| (ALUMINUM ZIRCONIUM | | |
| TETRACHLOROHYDREX GLY) | | |

An efficacy test is conducted by a panel of 5 experts:

The deodorant efficacy of each formula at 24 h and 48 h is evaluated after a single standardized application on the treated armpit compared to the untreated armpit by sensory evaluation (sniff-test).

The subjects have an average perspiration odor intensity on the armpits between 5 and 8 with a difference in intensity between the two armpits less than or equal to 1 (on a scale ranging from 0 to 10).

They perform 5 back-and-forth applications of composition under one armpit (average quantity deposited 0.40±0.05 g).

The results show that after a standardized application on the treated armpit versus the untreated armpit, a significant reduction in perspiration odor intensity is observed at 24 h and at 48 h for formula A.

The reductions observed are however not significantly different from the reductions observed with formulas C1 and C2.

In conclusion, the formula according to the invention is as effective as formulas comprising 15% aluminum salts (formula C1).

The combination of ingredients with a large quantity of starch, as is the case of formula A according to the invention, results in less greasy and drier sensory properties.

3/ Formula A according to the invention is also evaluated in terms of stick hardness, quantity deposited and stability (at 24 h at ambient temperature (AT), after 2 months at AT and after 2 months at 4° C.).

Stick hardness is measured according to the protocol described in the description.

Stick deposition was evaluated via an application on Wipall wipes (substrate mimicking armpit skin).

On each wipe, 5 stick passages are applied; 2 wipes are used for each formula to mimic both armpits. The quantity deposited is measured by weighing on a precision balance, before and after the 10 passages (it is estimated that a standard deposition is 0.4 g/armpit in vivo).

The appearance of the deposition is evaluated visually, in order to detect the presence of any white stains and/or particles.

The hardness values and quantities deposited are compared with two competitor products (1 and 2), which are commercial sticks based on natural raw materials.

The results are as follows:

TABLE 3

| | | Competitor | Competitor |
|---|---|---|---|
| | | product 1 | product 2 |
| Formulas | A | (comparison) | (comparison) |
| Hardness (N) | 9.32 | 7.69 | 3.39 |
| Deposition (g) | 0.6 | 0.6 | 1.0 |
| Stability: | | | |
| at 24 h at AT | Compliant | — | — |
| after 2 months at AT | Compliant | | |
| after 2 months at 4° C. | Compliant | | |

The deposited quantities are compliant and the quality of the depositions of both sticks according to the invention is better than the competitor products.

The formulas according to the invention thus offer the best compromise between stick stability, sufficient deposition, and deodorant performance.

### EXAMPLE 2

### Anhydrous Solid Compositions According to the Invention

Anhydrous solid compositions F1 to F7 according to the invention were prepared according to the protocol in example 1.

TABLE 4

| | F1 | F2 | F3 | |
|---|---|---|---|---|
| Ingredient | % (w/w) | % (w/w) | % (w/w) | Phase |
| CORN STARCH | 22 | 22 | 22 | C |
| MAGNESIUM OXIDE | 10 | 10 | 10 | B |
| CETYLSTEARYL ALCOHOL | 11.9 | 10.7 | 21.2 | A |
| REFINED RAPESEED OIL | Qs 100 | Qs 100 | Qs 100 | A |
| HYDROGENATED | 6.2 | 8 | 4.3 | A |
| CASTOR OIL | | | | |
| SHEA BUTTER | 4.6 | 4.1 | 3.2 | A |

TABLE 5

| | F4 | F5 | F6 | F7 | |
|---|---|---|---|---|---|
| Ingredient | % (w/w) | % (w/w) | % (w/w) | % (w/w) | Phase |
| CORN STARCH | 22 | 22 | 22 | 22 | C |
| MAGNESIUM OXIDE | 10 | 10 | 10 | 10 | B |
| CETYLSTEARYL | 15 | 15 | 15 | 15 | A |
| ALCOHOL | | | | | |
| REFINED | Qs | Qs | Qs | Qs | A |
| RAPESEED OIL | 100 | 100 | 100 | 100 | |
| CANDELILLA WAX | 4 | | | | A |
| SUNFLOWER WAX | | 4 | 10 | | A |
| RICE BRAN WAX | | | | 4 | A |
| SHEA BUTTER | 4.6 | 4.1 | 3.2 | 3.2 | A |

Formulas F1 to F3 according to the invention were packaged in plastic packaging, and formulas F1, F4 to F7 were packaged in cardboard packaging.

F1 to F3 according to the invention packaged in plastic packaging, and F1 packaged in cardboard packaging, were also evaluated in terms of stick hardness and deposited quantity:

TABLE 6

| | F1 | F1 | | |
|---|---|---|---|---|
| | (packaged | (packaged | | |
| | in plastic | in cardboard | | |
| Formulas | packaging) | packaging) | F2 | F3 |
| Hardness (N) | 14.73 | 7.85 | 12.63 | 24.49 |
| Deposition (g) | 0.5 | 0.5 | 0.5 | 0.35 |

The formulas according to the invention offer a good compromise between stick stability and sufficient deposition.

1. An anhydrous solid cosmetic composition comprising, in a cosmetically acceptable medium, from 5% to 25% by weight with respect to the total weight of the composition of at least one magnesium salt and from 15% to 40% by weight with respect to the total weight of the composition of starch.

2. The composition according to claim 1, wherein the magnesium salt is chosen from magnesium oxide, magnesium carbonate, magnesium hydroxide, magnesium bicarbonate and mixtures thereof

3. The composition according to claim 1, wherein the magnesium salt is magnesium oxide.

4. The composition according to claim 1, wherein the magnesium salt is present in a content ranging from 7 to 20% by weight, with respect to the total weight of the composition.

5. The composition according to claim 1, wherein the weight ratio between the magnesium salt(s) and the starch is less than or equal to 1.

6. The composition according to claim 1, comprises at least one fatty alcohol.

7. The composition according to claim 1, which comprises at least one wax chosen from hydrocarbon waxes; hydrogenated sunflower oil, hydrogenated castor oil, hydrogenated coconut oil, hydrogenated lanolin oil, and di-(trimethylo1-1,1,1 propane) tetrastearate.

8. The composition according to claim 1, which comprises at least one pasty compound chosen from isomerized jojoba oil, orange wax, cupuacu butter, shea butter, partially hydrogenated olive oil, cocoa butter, mango butter, shorea butter, murumuru butter, and mixtures thereof.

9. The composition according to claim 1, which comprises at least one oil.

10. The composition according to claim 1, which comprises less than 5% by weight of aluminum salt(s).

11. The composition according to claim 1, which is substantially free from silicone oil.

12. A product comprising a composition according to claim 1, packaged in a container made of plastic, cardboard, or paper.

13. A cosmetic method for treating body odors resulting from perspiration, comprising the application of a composition according to claim 1 on the skin.

14. The composition according to claim 2, wherein the magnesium salt is present in a content ranging from 7 to 20% by weight with respect to the total weight of the composition.

15. The composition according to claim 3, wherein the magnesium salt is present in a content ranging from 7 to 20% by weight with respect to the total weight of the composition.

16. The composition according to claim 2, wherein the weight ratio between the magnesium salt(s) and the starch is less than or equal to 1.

17. The composition according to claim 2, wherein the weight ratio between the magnesium salt(s) and the starch is less than or equal to 1.

18. The composition according to claim 3, wherein the weight ratio between the magnesium salt(s) and the starch is less than or equal to 1.

19. The composition according to claim 2, which comprises at least one fatty alcohol.

20. The composition according to claim 3, which comprises at least one fatty alcohol.
